(19) United States
(12) Patent Application Publication
(10) Pub. No.: US 2017/0246130 A1
(43) Pub. Date: Aug. 31, 2017

(54) 4-HYDROXYBENZOHYDARZIDE-A NEW CLASS OF ANGIOGENIC ENZYME THYMIDINE PHOSPHORYLASE INHIBITORS

(71) Applicants: Muhammad Iqbal Choudhary, Karachi (PK); Sumaira Javaid, Karachi (PK); Khalid M. Khan, Karachi (PK); Syed Muhammad Saad, Karachi (PK); Atia-tul Wahab, Karachi (PK); Atta-ur- Rahman, Karachi (PK)

(72) Inventors: Muhammad Iqbal Choudhary, Karachi (PK); Sumaira Javaid, Karachi (PK); Khalid M. Khan, Karachi (PK); Syed Muhammad Saad, Karachi (PK); Atia-tul Wahab, Karachi (PK); Atta-ur- Rahman, Karachi (PK)

(21) Appl. No.: 15/053,887

(22) Filed: Feb. 25, 2016

(51) Int. Cl. A61K 31/166

(52) U.S. Cl. CPC A61K 31/166

(57) ABSTRACT

The present invention relates to anti-thymidine phosphorylase compounds. These compounds are derivatives of 4-hydroxybenzohydarzide or generally Schiff bases of hydrazones. The invention evaluates a series of Schiff bases of hydrazones against thymidine phosphorylase, and identified significant inhibitors of thymidine phosphorylase enzyme during in vitro studies.

### BACKGROUND OF THE INVENTION

Low number, and unavailability of safer drugs for different pathological conditions is the key motivation for the continuous research in drug discovery and development. This process usually starts with disease target identification and validation, and then the discovery and identification of chemical compounds which could interact with the target. Chemical compounds found to be able to interact with target (lead molecules) then undergo the process of lead optimization. Lead molecules are subjected to toxicity studies, followed by pre-clinical and clinical studies.

The search for new chemical entities and a novel structural scaffold with therapeutic potential is therefore a key objective of pharmaceutical chemistry. Identification of lead molecules start from systematic synthesis of several privileged structures and their derivatives or screening of natural products (Hughes et al., 2010).

Angiogenesis is a primary physiological process for the fetal growth, during wound healing, and for female reproductive system. The process of angiogenesis involves endothelial cell (EC) activation, due to the binding of angiogenic molecules to the receptor present on ECs. This is followed by the release of various proteolytic enzymes which cause the degradation of basement membrane. Endothelial cells then undergo proliferation and migration towards the site which needs to get vascularised. With this, the process of formation of tube like structures starts. Finally, the maturation and stabilization of newly formed blood vessels takes place. This process has a significant involvement in a variety of pathological conditions, including cancer, rheumatoid arthritis, age-related macular degeneration, atherosclerosis, and diabetic retinopathy, etc. (Liekens et al., 2001). Aberrant angiogenesis is critical for cancer development. Its inhibition is therefore an important target for cancer chemotherapy. Various stages in angiogenesis cascade could be targeted to combat cancer. These include inhibition of proteolytic enzymes; inhibition of endothelial cell migration, proliferation and endothelial tube formation; inhibition of angiogenic growth factors; and inhibition of the angiogenic enzymes

In the present application, we employed an enzyme inhibition approach and targeted thymidine phosphorylase. The study of enzymes has a major significance as enzymes are vital for all life forms. Their under-expression or over-expression is implicated in a number of pathological conditions. Enzymes inhibition, associated with particular disease, is therefore an important approach for the treatment. The classical approach to develop an enzyme inhibitor is to mimic the structure of the substrate molecule. They are recognized mistakenly by the enzymes, and they ultimately lead to either blockage of the enzyme's catalytic activity or the production of non-functional products (Bjelakovic et al., 2002).

Enzyme thymidine phosphorylase (EC 2.4.2.4) is reported to be identical to human platelet derived endothelial cell growth factor (PD-ECGF). PD-ECGF is an intracellular, non-glycosylated protein. It is present in platelets, fibroblast, and transformed cell lines. This protein assists angiogenesis by facilitating endothelial cell migration, and proliferation. This protein is also termed angiogenic growth factor (Finnis et al., 1993). Thymidine phosphorylase (TP) enzyme is present in many cells and tissues. High levels of this enzyme are found in platelets, stromal cells, macrophages, endothelial cells, reticulocytes, glial cells, and ovary (De-Bruin et al., 2006).

Thymidine phosphorylase is comprised of two identical subunits, each subunit has 440 amino acids, with molecular weight of dimer ranges from 90 kDa-110 kDa in Escherichia coli and mammals, respectively. Each subunit consists of a large α/β domain and a smaller α-helical domain. These domains are separated from each other by a cleft or cavity (Walter et al., 1990).

The active site of Thymidine phosphorylase comprises thymidine and phosphate binding sites. It has been suggested that the products and substrates are produced as the anomeric carbon of sugar moves towards either the thymine nitrogen or the phosphate oxygen, while the latter two species showed only minimal movement, as shown in Figure-1. α-2-D-Deoxyribose-1-phosphate (2DDR-1P) then undergo dephosphorylation to produce 2-D-deoxyribose, which is used either as an energy source for the cell or secreted out of the cell where it may act as an angiogenic growth factor (Reigan et al., 2005). Kinetic studies on E. coli TP showed that enzyme followed bi-bi (sequential) mechanism where phosphate is the first substrate to interact with the enzyme, and 2DDR-1P is the last reaction product to dissociate from the enzyme (Bronckaers et al., 2009; Pugmire et al., 1998). This enzyme also possesses transferase activity, which is used to transfer the deoxyribosyl moiety between the two pyrimidine bases (Bronckaers et al., 2009).

Thymidine phosphorylase plays an important role in controlling nucleic acid homeostasis by ensuring the proper supply of dNTPs during replication/repair of DNA. With high levels in blood platelets, placenta, and endometrium, thymidine phosphorylase also has a very important function in wound healing, and the reproductive cycle of females (Bronckaers et al., 2009). It also plays a role in neuronal cells survival. thymidine phosphorylase overexpression has been reported in patients with rheumatoid arthritis (in their synovial fluid), chronic inflammatory diseases, psoriasis, and tumor angiogenesis. Neoplastic tissues of bladder, gastric, cervical, lung, colon, esophageal, and breast cancers showed a higher expression of Thymidine phosphorylase (De-Bruin et al., 2006).

Over-expression of thymidine phosphorylase in tumor cells of a number of organs is an indication of its role in angiogenesis. It is an important regulator of angiogenic function of endothelial progenitor cells (EPC). These progenitor cells can differentiate into endothelial cells (EC) which are the key players in angiogenesis. EPC also has the capacity to invade tumor mass, and differentiate into EC which then facilitate tumor angiogenesis and metastasis. Thymidine phosphorylase and degradation product of thymidine phosphorylase metabolites 2-deoxy-D-ribose-1-phosphate (2DDR-1P) and thymine (i.e., 2DDR and β-amino-iso-butyric acid, respectively) facilitate angiogenesis by inducing EC migration, and EC tube formation in vitro and in vivo (Bronckaers et al., 2009, Brown and Bicknell 1998).

Thymidine phosphorylase is an important angiogenic molecule. Unlike other angiogenic molecules it is not released into extracellular space to cause endothelial cell activation. It actually lacks amino terminal hydrophobic leader sequence which helps in the secretion of protein into extracellular space. Its metabolite i.e., 2DDR, however, can be released in extracellular space where it can exert its angiogenic effect. Some tumor cell lines do secret thymidine phosphorylase into extracellular space after its post-translational modification where its serine residues covalently attaches to nucleotide's phosphate groups, generating nucleotidylated protein which can be released into the extracellular space. In addition to this, thymidine phosphorylase and 2DDR, unlike other angiogenic factors, facilitates angiogenesis via non receptor mediated mechanism as mammalian cell do not possess receptors for these molecules (Bronckaers et al., 2009; Brown and Bicknell, 1998).

Significant efforts have been focused on the development of thymidine phosphorylase inhibitors with possible therapeutic potential since the 1960s. Some of these molecules were identified with excellent thymidine phosphorylase inhibitory activity, and were tested pre-clinically and clinically, but currently no product is approved for clinical use (Bronckaers et al., 2009). Possible reasons for the failure of drug to approve for clinical use includes poor pharmacokinetics of drug, increased drug efflux rate or limited uptake of drug by the biological system and most importantly severe side effects associated with inhibitor. Therefore, there is a need to identify new and effective anti-thymidine phosphorylase compounds which can be used as anti-angiogenic drugs. These can be further investigated for some other angiogenic studies, and for pre-clinical and clinical studies.

The present invention relates to anti-thymidine phosphorylase compounds. These compounds are derivatives of 4-hydroxybenzohydarzide or generally Schiff bases of hydrazones. Schiff bases are nitrogen analogues of ketones or aldehydes in which the carbonyl moiety is replaced by an azomethine or imine group. Hydrazones on the other side are also related to ketones and aldehydes with the structure of —CONHN═CH— group. Schiff bases and hydrazones were reported to possess a wide variety of biological properties. Some of them are anticonvulsant, antibacterial, anti-hypertensive, anti-inflammatory, anti-fungal, anticancer, antipyretic, antimicrobial, cytotoxic activity, anti-HIV, and herbicidal activities (Ananad et al., 2012; Padmini et al., 2013). The combination of hydrazones with Schiff bases leads to compounds with unique biochemical character. Here, we have evaluated a series of Schiff bases of hydrazones against thymidine phosphorylase, and identified as significant inhibitors of thymidine phosphorylase enzyme during in vitro studies.

### BRIEF SUMMARY OF THE INVENTION

Angiogenesis is a primary physiological process for the fetal growth, during wound healing, and for female reproductive system. Process of angiogenesis involves the endothelial cell activation, followed by the degradation of basement membrane. endothelial cells then undergo proliferation and migration towards the site which needs to be vascularised. Endothelial cells then undergo the process of formation of tube like structures (new blood vessel sprout). Finally the maturation and stabilization of newly formed blood vessel takes place. Apart from physiological role, this process also has a significant involvement in a variety of pathological conditions (Liekens et al., 2001). Aberrant angiogenesis is critical for cancer development. Targeting angiogenesis is thus an important anti-cancer approach. Various stages in angiogenesis (such as secretion of proteolytic enzymes, endothelial cell migration, proliferation and endothelial tube formation or angioenic enzymes) can be targeted to control cancer progression.

In the present application, we have used an enzyme inhibition approach and targeted an angiogenic enzyme i.e., thymidine phosphorylase. This is an enzyme of pyrimidine salvage pathway mainly responsible for ensuring nucleotide homeostasis. This enzyme has been reported to be involved in a number of pathological conditions described earlier (De-Bruin et al., 2006). Role of thymidine phosphorylase as angiogenic molecule/enzyme was proposed after the finding that it is identical to PD-ECGF which promotes angiogenesis by facilitating endothelial cell proliferation, and migration. It is an important regulator of angiogenic potential of endothelial progenitor cells (EPC). These progenitor cells can differentiate into endothelial cells (key players of angiogenesis). Products of TP reaction facilitate angiogenesis by inducing EC migration, and EC tube formation in vitro and in vivo (Bronckaers et al., 2009, Brown and Bicknell 1998).

### BRIEF DESCRIPTION OF DRAWING

FIG. 1 depicts mechanism of reaction, catalyzed by enzyme thymidine phosphorylase, substrate thymidine and phosphate react to produce thymine and 2-deoxy-D-ribose-1-phosphate.

FIG. 2 depicts the basic skeleton of 4-hydroxybenzohydrazide derivatives 1-30.

FIG. 3 depicts Lineweaver-burk plot of compound 24 in which reciprocal of substrate concentration (1/S) is plotted on x-axis, while reciprocal of rate of reaction (1/v) is plotted on y-axis in the absence and different concentrations of compound 24. FIG. 3 shows that apparent km of the enzyme remains unaffected while the apparent Vmax decreased.

FIG. 4 depicts secondary replot of Lineweaver-Burk plot between the slopes (Km/Vmax) of each line on lineweaver-burk plot versus different concentrations of compound 24.

FIG. 5 depicts Dixon plot of reciprocal of rate of reaction (velocities) versus different concentrations of compound 24.

FIG. 6 depicts Lineweaver-burk plot of compound 27 in which reciprocal of substrate concentration (1/S) is plotted on x-axis, while reciprocal of rate of reaction (1/v) is plotted on y-axis in the absence and different concentrations of compound 27. FIG. 6 shows that apparent km of the enzyme remains unaffected while the apparent Vmax decreased.

FIG. 7 depicts secondary replot of Lineweaver-Burk plot between the slopes (Km/Vmax) of each line on lineweaver-burk plot versus different concentrations of compound 27.

FIG. 8 depicts principle of MTT assay, in which MTT is converted into formazan by mitochondrial enzyme succinate dehydrogenase.

### DETAILED DESCRIPTION OF THE INVENTION

In the present application, we have studied the inhibitory activity of derivatives of 4-hydroxybenzohydarzide via in-vitro studies. We performed the primary studies on thirty derivatives of 4-hydroxybenzohydarzide (Table 1) by using spectrphotometric thymidine phosphorylase inhibition protocol. In this study we first identify active compounds capable of inhibiting the TP enzyme (Table-II). Some of the most active compounds (among the series) were then subjected to kinetic studies in order to find out their mechanism of inhibition and to evaluate the kinetic parameters. These compounds showed non-competitive and uncompetitive modes of inhibition (Table-II).

TABLE 1
In-vitro thymidine phosphorylase inhibitory activities of derivatives of 4-
hydroxybenzohydarzides.

| Compound | Structure | IC₅₀ (μM ± S.E.M.) |
|---|---|---|
| 1 | 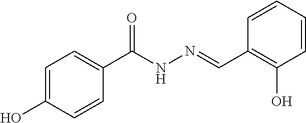 [SMILES: O=C(N/N=C/c1ccccc1O)c1ccc(O)cc1] | 189.1 ± 0.1 |
| | 4-Hydroxy-N′-[(E)-(2- | |
| | hydroxyphenyl)methylidene]benzohydrazide | |
| 2 | 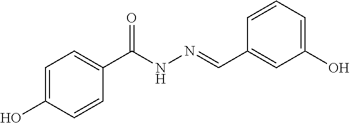 [SMILES: O=C(N/N=C/c1cccc(O)c1)c1ccc(O)cc1] | 180 ± 3.0 |
| | 4-Hydroxy-N′-[(E)-(3- | |
| | hydroxyphenyl)methylidene]benzohydrazide | |
| 3 | 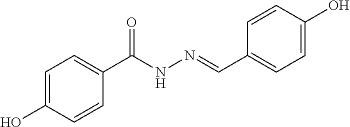 [SMILES: O=C(N/N=C/c1ccc(O)cc1)c1ccc(O)cc1] | 199.4 ± 0.4 |
| | 4-Hydroxy-N′-[(E)-(4- | |
| | hydroxyphenyl)methylidene]benzohydrazide | |
| 4 | 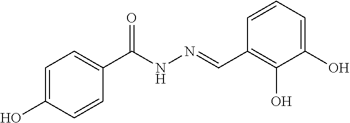 [SMILES: O=C(N/N=C/c1cccc(O)c1O)c1ccc(O)cc1] | 170.6 ± 0.5 |
| | 4-Hydroxy-N′-[(E)-(2,3- | |
| | dihydroxyphenyl)methylidene]benzohydrazide | |
| 5 | 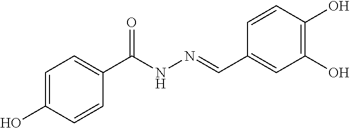 [SMILES: O=C(N/N=C/c1ccc(O)c(O)c1)c1ccc(O)cc1] | 229.5 + 2.1 |
| | 4-Hydroxy-N′-[(E)-(3,4- | |
| | dihydroxyphenyl)methylidene]benzohydrazide | |
| 6 | 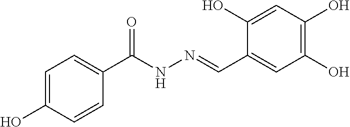 [SMILES: O=C(N/N=C/c1cc(O)c(O)cc1O)c1ccc(O)cc1] | 208.0 ± 0.9 |
| | 4-Hydroxy-N′-[(E)-(2,4,5- | |
| | trihydroxyphenyl)methylidene]benzohydrazide | |
| 7 | 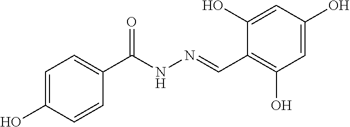 [SMILES: O=C(N/N=C/c1c(O)cc(O)cc1O)c1ccc(O)cc1] | Inactive |
| | 4-Hydroxy-N′-[(E)-(2,4,6- | |
| | trihydroxyphenyl)methylidene]benzohydrazide | |
| 8 | 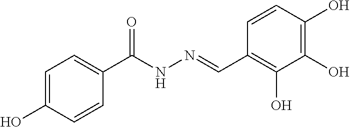 [SMILES: O=C(N/N=C/c1ccc(O)c(O)c1O)c1ccc(O)cc1] | Inactive |
| | 4-Hydroxy-N′-[(E)-(2,3,4- | |
| | trihydroxyphenyl)methylidene]benzohydrazide | |
| 9 | 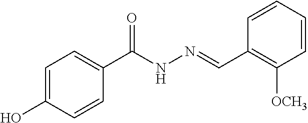 [SMILES: COc1ccccc1/C=N/NC(=O)c1ccc(O)cc1] | 183.7 ± 1.5 |
| | 4-Hydroxy-N′-[(E)-(2- | |
| | methoxyphenyl)methylidene]benzohydrazide | |
| 10 | 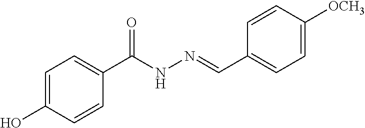 [SMILES: COc1ccc(/C=N/NC(=O)c2ccc(O)cc2)cc1] | 185.5 ± 2.0 |
| | 4-Hydroxy-N′-[(E)-(4- | |
| | methoxyphenyl)methylidene]benzohydrazide | |
| 11 | 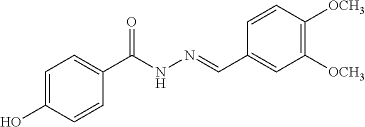 [SMILES: COc1ccc(/C=N/NC(=O)c2ccc(O)cc2)cc1OC] | 204.1 ± 3.0 |
| | 4-Hydroxy-N′-[(E)-(3,4- | |
| | dimethoxyphenyl)methylidene]benzohydrazide | |
| 12 | 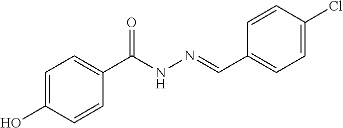 [SMILES: O=C(N/N=C/c1ccc(Cl)cc1)c1ccc(O)cc1] | 159.0 ± 0.4 |
| | 4-Hydroxy-N′-[(E)-(4- | |
| | chlorophenyl)methylidene]benzohydrazide | |
| 13 | 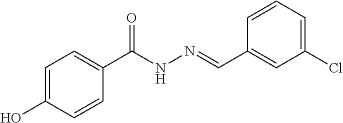 [SMILES: O=C(N/N=C/c1cccc(Cl)c1)c1ccc(O)cc1] | 160.3 ± 1.0 |
| | 4-Hydroxy-N′-[(E)-(3- | |
| | chlorophenyl)methylidene]benzohydrazide | |
| 14 | 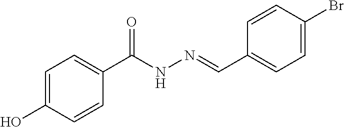 [SMILES: O=C(N/N=C/c1ccc(Br)cc1)c1ccc(O)cc1] | 158.0 ± 1.0 |
| | 4-Hydroxy-N′-[(E)-(4- | |
| | bromophenyl)methylidene]benzohydrazide | |
| 15 | 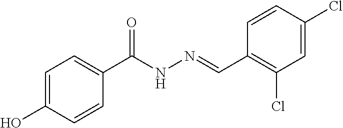 [SMILES: O=C(N/N=C/c1ccc(Cl)cc1Cl)c1ccc(O)cc1] | Inactive |
| | 4-Hydroxy-N′-[(E)-(2,4- | |
| | dichlorophenyl)methylidene]benzohydrazide | |
| 16 | 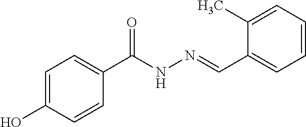 [SMILES: Cc1ccccc1/C=N/NC(=O)c1ccc(O)cc1] | 177.2 ± 0.5 |
| | 4-Hydroxy-N′-[(E)-(2- | |
| | methylphenyl)methylidene]benzohydrazide | |
| 17 | 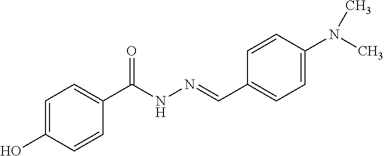 [SMILES: CN(C)c1ccc(/C=N/NC(=O)c2ccc(O)cc2)cc1] | Inactive |
| | 4-Hydroxy-N′-[(E)-[4- | |
| | dimethylaminophenyl]methylidene]benzohydrazide | |
| 18 | 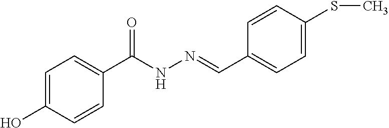 [SMILES: CSc1ccc(/C=N/NC(=O)c2ccc(O)cc2)cc1] | Inactive |
| | 4-Hydroxy-N′-[(E)-[4- | |
| | (methylsulfanylphenyl)methylidene]benzohydrazide | |
| 19 | 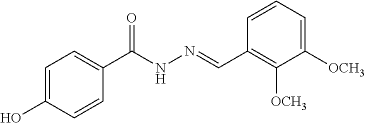 [SMILES: COc1cccc(/C=N/NC(=O)c2ccc(O)cc2)c1OC] | 181.5 ± 1.6 |
| | 4-Hydroxy-N′-[(E)-(2-hydroxy-3- | |
| | methoxyphenyl)methylidene]benzohydrazide | |
| 20 | 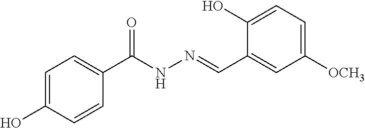 [SMILES: COc1ccc(O)c(/C=N/NC(=O)c2ccc(O)cc2)c1] | 172.0 ± 2.7 |
| | 4-Hydroxy-N′-[(E)-(2-hydroxy-5- | |
| | methoxyphenyl)methylidene]benzohydrazide | |
| 21 | 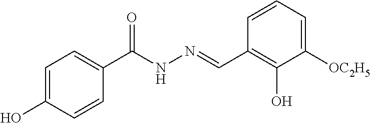 [SMILES: CCOc1cccc(/C=N/NC(=O)c2ccc(O)cc2)c1O] | 190.3 ± 0.4 |
| | 4-Hydroxy-N′-[(E)-(3-ethoxy-2- | |
| | hydroxyphenyl)methylidene]benzohydrazide | |
| 22 | 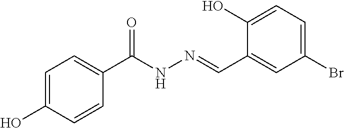 [SMILES: O=C(N/N=C/c1cc(Br)ccc1O)c1ccc(O)cc1] | Inactive |
| | 4-Hydroxy-N′-[(E)-(5-bromo-2- | |
| | hydroxyphenyl)methylidene]benzohydrazide | |
| 23 | 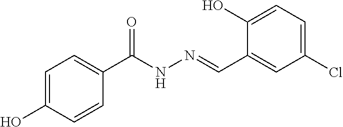 [SMILES: O=C(N/N=C/c1cc(Cl)ccc1O)c1ccc(O)cc1] | Inactive |
| | 4-Hydroxy-N′-[(E)-(5-chloro-2- | |
| | hydroxyphenyl)methylidene]benzohydrazide | |
| 24 | 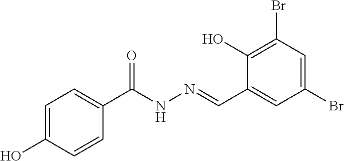 [SMILES: O=C(N/N=C/c1cc(Br)cc(Br)c1O)c1ccc(O)cc1] | 6.8 ± 0.7 |
| | 4-Hydroxy-N′-[(E)-(3,5-dibromo-2- | |
| | hydroxyphenyl)methylidene]benzohydrazide | |
| 25 | 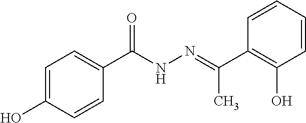 [SMILES: C/C(=N\NC(=O)c1ccc(O)cc1)c1ccccc1O] | Inactive |
| | 4-Hydroxy-N′-[(E)-1-(2- | |
| | Hydroxyphenyl)ethylidene]benzohydrazide | |
| 26 | 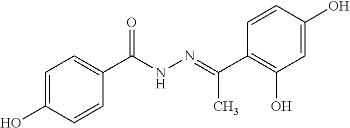 [SMILES: C/C(=N\NC(=O)c1ccc(O)cc1)c1ccc(O)cc1O] | Inactive |
| | 4-Hydroxy-N′-[(E)-1-(2,4- | |
| | dihydroxyphenyl)ethylidene]benzohydrazide | |
| 27 | 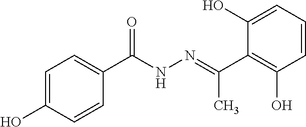 [SMILES: C/C(=N\NC(=O)c1ccc(O)cc1)c1c(O)cccc1O] | 176.9 ± 1.6 |
| | 4-Hydroxy-N′-[(E)-1-(2,6- | |
| | dihydroxyphenyl)ethylidene]benzohydrazide | |
| 28 | 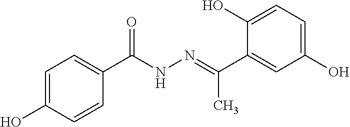 [SMILES: C/C(=N\NC(=O)c1ccc(O)cc1)c1cc(O)ccc1O] | 183.4 ± 0.9 |
| | 4-Hydroxy-N′-[(E)-1-(2,5- | |
| | dihydroxyphenyl)ethylidene]benzohydrazide | |
| 29 | 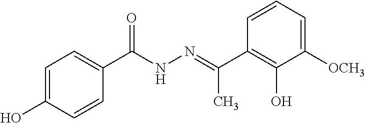 [SMILES: COc1cccc(/C(C)=N/NC(=O)c2ccc(O)cc2)c1O] | 174.2 ± 1.0 |
| | 4-Hydroxy-N′-[(E)-1-(2-hydroxy-3- | |
| | methoxyphenyl)ethylidene]benzohydrazide | |
| 30 | 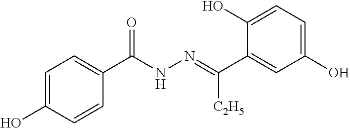 [SMILES: CC/C(=N\NC(=O)c1ccc(O)cc1)c1cc(O)ccc1O] | 173.0 ± 1.4 |
| | 4-Hydroxy-N′-[(E)-1-(2,5- | |
| | dihydroxyphenyl)propylidene]benzohydrazide | |
| Standard Compound (7-Deazaxanthine) | 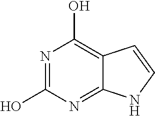 [SMILES: Oc1nc(O)c2cc[nH]c2n1] | 41 ± 1.63 |
| | 2,4-Dihydroxypyrrolo[2,3-d]pyrimidine 7H- | |
| | Pyrrolo [2,3-d]pyrimidine-2,4-diol | |

SEM^{a} is the Standard Error of the Mean;
N.A.^{b} means Not Active

Compounds active against TP, were also evaluated for their effect on the proliferation of fibroblast cells (3T3 cells) and cancerous cells (e.g. PC3). Some compounds in addition of inhibiting the angiogenic enzyme TP were able to significantly inhibit the proliferation of 3T3 cells and PC3 cancer cells (Table-III).

TABLE II
Kinetic studies of active compounds on thymidine phosphorylase.

| Compound | Ki^{a} (μM ± S.E.M.)^{b} | Inhibition Type |
|---|---|---|
| 4-Hydroxy-N′-[(E)-(3,4- | 176.65 ± 0.006 | Uncompetitive |
| dihydroxyphenyl) | | |
| methylidene]benzohydrazide (5) | | |
| 4-Hydroxy-N′-[(E)-(4- | 138.0 ± 0.009 | Uncompetitive |
| chlorophenyl) | | |
| methylidene]benzohydrazide | | |
| (12) | | |
| 4-Hydroxy-N′-[(E)-(4- | 80.5 ± 0.002 | Uncompetitive |
| bromophenyl) | | |
| methylidene]benzohydrazide | | |
| (14) | | |
| 4-Hydroxy-N′-[(E)-(3,5- | 1.75 ± 0.009 | Non-Competitive |
| dibromo-2-hydroxyphenyl) | | |
| methylidene]benzohydrazide | | |
| (24) | | |
| 4-Hydroxy-N′-[(E)-1-(2,6- | 168.0 ± 0.003 | Uncompetitive |
| dihydroxyphenyl) | | |
| ethylidene]benzohydrazide (27) | | |
| 4-Hydroxy-N′-[(E)-1-(2,5- | 159.05 ± 0.002 | Uncompetitive |
| dihydroxyphenyl) | | |
| propylidene]benzohydrazide | | |
| (30) | | |
| 7-De-azaxanthine (Standard) | 45.66 ± 0.0009 | Non-Competitive |

Ki^{a} Dissociation constant,
SEM^{b} is the Standard Error of the Mean

TABLE III
In-vitro Anti-proliferative activities of active compounds

| | IC₅₀ ± SD (μM)^{a} | |
|---|---|---|
| Compound | 3T3 Cell Line | PC3 Cell Line |
| 4-Hydroxy-N′-[(E)-(2-hydroxyphenyl) | 13.1 ± 0.3 | 7.579 ± 0.462 |
| methylidene]benzohydrazide (1) | | |
| 4-Hydroxy-N′-[(E)-(3-hydroxyphenyl) | Inactive | Inactive |
| methylidene]benzohydrazide (2) | | |
| 4-Hydroxy-N′-[(E)-(4-hydroxyphenyl) | Inactive | Inactive |
| methylidene]benzohydrazide (3) | | |
| 4-Hydroxy-N′-[(E)-(2,3-dihydroxyphenyl) | 23.0 ± 1.0 | 7.611 ± 0.4898 |
| methylidene]benzohydrazide (4) | | |
| 4-Hydroxy-N′-[(E)-(3,4-dihydroxyphenyl) | 23.5 ± 1.1 | Inactive |
| methylidene]benzohydrazide (5) | | |
| 4-Hydroxy-N′-[(E)-(2,4,5-trihydroxyphenyl) | 3.8 ± 0.4 | Inactive |
| methylidene]benzohydrazide (6) | | |
| 4-Hydroxy-N′-[(E)-(2-methoxyphenyl) | Inactive | Inactive |
| methylidene]benzohydrazide (9) | | |
| 4-Hydroxy-N′-[(E)-(4-methoxyphenyl) | Inactive | Inactive |
| methylidene]benzohydrazide (10) | | |
| 4-Hydroxy-N′-[(E)-(3,4-dimethoxyphenyl) | Inactive | Inactive |
| methylidene]benzohydrazide (11) | | |
| 4-Hydroxy-N′-[(E)-(4-chlorophenyl) | Inactive | Inactive |
| methylidene]benzohydrazide (12) | | |
| 4-Hydroxy-N′-[(E)-(3-chlorophenyl) | Inactive | Inactive |
| methylidene]benzohydrazide (13) | | |
| 4-Hydroxy-N′-[(E)-(4-bromophenyl) | Inactive | Inactive |
| methylidene]benzohydrazide (14) | | |
| 4-Hydroxy-N′-[(E)-(2-methylphenyl) | Inactive | Inactive |
| methylidene]benzohydrazide (16) | | |
| 4-Hydroxy-N′-[(E)-(2-hydroxy-3- | 23.6 ± 0.2 | 9.6114 ± 0.394 |
| methoxyphenyl)methylidene]benzohydrazide (19) | | |
| 4-Hydroxy-N′-[(E)-(2-hydroxy-5-methoxyphenyl) | 15.3 ± 0.9 | 10.507 ± 0.5506 |
| methylidene]benzohydrazide (20) | | |
| 4-Hydroxy-N′-[(E)-(3-ethoxy-2-hydroxyphenyl) | 9.4 ± 0.3 | 6.5425 ± 0.2775 |
| methylidene]benzohydrazide (21) | | |
| 4-Hydroxy-N′-[(E)-(3,5-dibromo-2- | 9.2 ± 0.1 | 8.338 ± 0.965 |
| hydroxyphenyl) methylidene]benzohydrazide (24) | | |
| 4-Hydroxy-N′-[(E)-1-(2,6-dihydroxyphenyl) | 26.7 ± 1.0 | Inactive |
| ethylidene]benzohydrazide (27) | | |
| 4-Hydroxy-N′-[(E)-1-(2,5- | 20.2 ± 0.9 | Inactive |
| dihydroxyphenyl)ethylidene]benzohydrazide (28) | | |
| 4-Hydroxy-N′-[(E)-1-(2-hydroxy-3- | 18.6 ± 0.6 | 8.485 ± 0.592 |
| methoxyphenyl) ethylidene]benzohydrazide (29) | | |
| 4-Hydroxy-N′-[(E)-1-(2,5- | 19.5 ± 0.9 | Inactive |
| dihydroxyphenyl)propylidene]benzohydrazide (30) | | |
| Reference Compound (Cyclohexamide) | 0.26 ± 0.1 μM | — |
| Reference Compound (Doxorubucin) | — | 0.91 ± 0.1 μM |

SD^{a} is the Standard Deviation

The present invention identifies compounds which are potentially useful as anti-angiogenic agents, as they showed a potent in-vitro inhibitory activity against angiogenic enzyme, thymidine phosphorylase. Some compounds in addition to inhibiting the angiogenic enzyme TP were able to significantly inhibit the proliferation of 3T3 cells, and PC3 cancer cells. Proliferation (as stated earlier) is an important step of angiogenesis process to occur.

### In-Vitro Thymidine Phosphorylase Inhibition Assay

Total thirty analogues of 4-hydroxybenzohydarzide were evaluated. Out of which 21 compounds were found to be active against TP. Among them, compound 24 showed a potent TP inhibitory activity (IC₅₀=6.8-0.7 μM). While other active compounds showed a moderate inhibition towards thymidine phosphorylase (Table-I), in comparison of standard compound used i.e. 7-deazaxanthine (IC₅₀=41.0±1.63 μM).

Eight derivatives with mono, di and tri OH substitutions were evaluated and six were found to be active against the TP enzyme with IC50 value ranges between 170.6-229.5 μM. Among monohydroxylated derivatives, compounds 1, 2, and 3 with OH at ortho, meta, and para positions on phenyl ring, respectively, showed IC50=189.1±0.1, 180.0±3.0, and 199.4±0.4 μM, respectively. Among di-hydroxylated derivatives, compound 4 with two OH at ortho and meta positions showed moderate TP inhibition (IC₅₀=170.6±0.5 μM), while compound 5 with two OH at para and meta positions also showed weak TP inhibition (IC50=229.5±0.4 μM). Among tri-hydroxylated derivatives compound 6 with three OH at ortho (1′), meta (3′), and para (4′) positions showed a weak TP inhibition (IC₅₀=208.0±0.9 μM), while compound 7 with two OH at ortho, and one at para positions and compound 8 with three OH at ortho (1′), meta (2′), and para (3′) positions on phenyl ring were found to be inactive.

Structure-activity relationship (SAR) proposed that hydroxyl substitutions on phenyl ring at different position plays an important role in inducing thymidine phosphorylase inhibition. The ability of inhibiting thymidine phosphorylase was found to be more in monohydroxylated derivatives than di or tri-hydroxylated derivatives. OH groups being strong electron donating group might enhances the ability of phenyl ring to undergo hydrogen bonding and π-π interactions with the amino acid residues, present in the active site or hydrophobic pocket of the TP enzyme.

Three derivatives with mono, and di-OCH₃ substitutions were evaluated and found to be active against TP enzyme with IC₅₀ value ranges between 183.7-204.1 μM. Among monomethoxylated derivatives, compounds 9, and 10 with OCH₃ at ortho, and para positions on phenyl ring showed a moderate TP inhibition (IC₅₀=183.7±1.5 and 185.5±2.0 μM, respectively). Compound 11 with two OCH₃ at para and meta positions showed a weak TP inhibition with IC₅₀=204.1±3.0 μM).

SAR proposed that monomethoxylated derivatives were slightly more effective in inhibiting TP than dimethoxylated derivative based on their IC₃₀ values. OCH₃ group being electron donating groups were proposed to be involved in hydrogen bonding and hydrophobic interaction with residues present in the active site or hydrophobic pocket of TP.

Four derivatives with mono, and di-halogen substitutions were evaluated and three were found to be active against TP enzyme with IC₅₀ value ranges between 159.0-160.3 M. Among monohalogenated derivatives, compounds 12, and 13 with Cl at para, and meta positions and compound 14 with Br at para position on phenyl ring showed moderate TP inhibition (IC₅₀=159.0±0.4, 160.3±1.0, and 158.0±1.0 μM, respectively). Compound 15 with two Cl groups at ortho, and para positions were found to be inactive.

SAR proposed that monohalogenated substitution were more capable of inducing TP inhibition, while dihalogenated substitutions found unable to inhibit enzyme. It was proposed that being electron donating (when present on benzene ring), halogens increase the ability of these compounds to interact hydrophobically via π-π interactions with residues present in the active site or hydrophobic pocket of TP.

Compound 16 with methyl group at ortho position on phenyl ring showed a moderate inhibition of TP (IC₅₀=177.2±0.5 μM), methyl being electron donating group proposed to increase the hydrophobic interactions of the compound with TP enzyme. Compounds 17 and 18 with dimethylmine and sulfmethyl groups, attached to para positions on phenyl ring were unable to inhibit the enzyme.

Three derivatives with hydroxyl-cum-methoxy substitutions were evaluated and all were found to be active against TP enzyme with IC₅₀ values between 172.0-190.3 μM. Compound 19 with OCH₃ and OH group at meta (4′) and ortho (5′) positions, respectively, showed a moderate TP inhibition (IC₃₀=181.5±1.6). Replacement of OCH₃ and OH groups at meta (4′) and ortho (1′) positions, respectively, in compound 20 slightly increased the inhibition of TP (IC₅₀=172.0±2.7 μM). Replacement of OCH₃ (in compound 19) with OC₂H₅ group in compound 21 slightly decreased the TP inhibition (IC₅₀=190.3±0.4 μM).

SAR proposed that when OH and OCH₃ groups were present consecutively they lower the ability of compounds to inhibit enzyme, as revealed by their IC₅₀ values. OH and OCH₃ electron donating groups were proposed to be involved in hydrogen bonding and hydrophobic interactions with residues present in the active site or hydrophobic pocket of TP.

Three derivatives with hydroxyl-cum-halogen substitutions were evaluated and only one compound showed a potent inhibitory activity against TP enzyme. Compound 22 with Br at meta and OH at ortho positions, and compound 23 with Cl substitution at meta and OH at ortho positions on phenyl ring were found to be inactive. Compound 24 with two Br groups at meta positions and OH at ortho positions of phenyl ring showed potent TP inhibitory activity (IC₅₀=6.8±0.7 μM), in comparison to the standard used i.e., 7-DX (IC₅₀=41.0±1.63 M). SAR proposed that presence of two bromine groups along with a hydroxyl group make compound very potent. OH groups being strong electron donating might enhance the ability of phenyl ring to undergo hydrogen bonding/π-π interactions with the amino acid residues. Halogens on the other hand increase the ability of these compounds to interact hydrophobically via π-π interactions with residues present in the active site or hydrophobic pocket of TP.

The next derivitization was made by replacing the benzylidene group with ethylidine group (FIG. 2). In addition of this, OH and OCH₃ groups were also substituted on phenyl ring. Compound 25 with single OH group at ortho position on phenyl ring was found to be inactive. Among di-hyrdroxylated derivatives, compound 26 with two OH at ortho and para positions, on phenyl ring was found to be inactive. Compound 27 with two OH groups on ortho positions and compound 28 with two OH groups on ortho and meta positions on phenyl ring showed a moderate inhibition of TP (IC₅₀=176.9±1.6 and 183.4±0.9 μM, respectively). Compound 29 with methoxy and hydroxyl groups at meta and ortho positions on phenyl ring, respectively, showed moderate TP inhibition (IC₅₀=174.2±1.0 μM).

The next derivitization was made by replacing the benzylidene group with propylidine group (2). In addition of this, OH group was also substituted on phenyl ring. Compound 30 with two hydroxyls on ortho and meta positions of phenyl ring showed a moderate TP inhibition (IC₅₀=173.0±1.4 M).

### Mechanism Based Studies of Active Compounds

In order to determine the type of inhibition of most active compounds, kinetic studies were carried out. These studies disclose the mechanism of inhibitor binding to enzyme. Kinetic studies were carried out using thymidine as variable substrate. Kinetic study on compound 24 showed that it inhibits thymidine phosphorylase enzymes in a non-competitive manner (Table-II, Figure-4). This was deduced from Lineweaver Burk plot. This compound therefore, interacted either with the amino acids of hydrophobic pocket of the enzyme or at allosteric site of the enzyme. Kinetic study on compounds 5, 12, 14, 27, and 30 revealed that they inhibited thymidine phosphorylase in un-competitive manner (Table-II). Uncompetitive inhibitors encountered enzyme only when enzyme-substrate complex has been formed, enzyme-substrate complex formation was proposed to induce a conformational changes in the enzyme which facilitate the binding of inhibitor. Uncompetitive inhibitor causes decrease in both Km and Vmax values of the enzyme (Figure-6). Non-competitive inhibitor does not affect the Km value but it changes the Vmax value. Ki values were determined by secondary re-plot, and confirmed by Dixon plot. Mode of inhibition and Ki values are given in Table-II.

### Anti-Proliferative Studies of Active Compounds

Compounds found to be active in inhibiting thymidine phosphorylase were then subjected to MTT proliferation assay. Rapid proliferation is the property inherent to cancerous/tumor cells. This assay was carried out to study the effect of these compounds on proliferation of various cell lines. The cell lines which we targeted were mouse fibroblast cell line (3T3), and prostate cancer line (PC3).

Compounds 1, 4, 5, 6, 19, 20, 21, 24, 27, 28, 29, and 30 were able to significantly inhibit the proliferation of 3T3 cells (Table-III), in comparison to reference compound i.e., cyclohexamide (IC₅₀=0.26±0.1 M). Compounds 1, 4, 19, 20, 21, 24, and 29 were able to significantly inhibit the proliferation of PC3 cancer cells (Table-III) in comparison of reference compound i.e., doxorubucin (IC₅₀=0.91±0.1 μM). These compounds therefore also possess anti-proliferative activity against 3T3 and PC3 cells.

### Material and Methods

Chemicals for In Vitro Thymidine Phosphorylase Studies:

Enzyme thymidine phosphorylase (E. coli) and its substrate thymidine were purchased from Sigma Aldrich, USA, and inhibitor 7-deazaxanthine was purchased from Santa Cruze Biotechnology Inc., USA. In vitro studies on thymidine phosphorylase were carried out in 96-well microtiter plate using Spectra Max-340 and Spectra Max-384 spectrophotometers (Molecular Devices, CA, USA). Deionized water used for buffer preparation was collected by Simplicity Water Purification System (Milipore).

### Chemicals Used for Proliferative Studies (MTT Assay):

Dulbecco's modified eagle medium (DMEM), cyclohexamide and doxorubucin were purchased from Sigma Aldrich, USA, mouse fibroblast cell line (3T3), and prostate cancer line (PC3) were purchased from American Type Culture Collection (ATCC), USA, 0.25% Trypsin EDTA was purchased from Gibco, Invitrogen, New Zealand, fetal bovine serum (FBS) was purchased from A&E Scientific (PAA), USA, 0.4% Trypan Blue solution was purchased from Amersco, USA, 3-(4,5-dimethythiazol-2-yl)-2,5-diphenyl tetrazolium bromide (MTT) was purchased from MP Biomedicals, France.

### Thymidine Phosphorylase Assay

Since human TP is not commercially available, we used commercially available recombinant E. coli TP (expressed in Escherichia coli) enzyme. Primary sequence of TP is mostly conserved throughout the evolution as mammalian TP share 39% sequence similarity with TP of E. coli. The mammalian enzyme also shared 65-70% similarity with the active site residues of E. coli TP enzyme (Bronckaers et al., 2009). Assay for thymidine phosphorylase inhibition was performed spectrophotometrically, following method of Bera et al. with some modifications (Bera et al., 2013).

### Principle

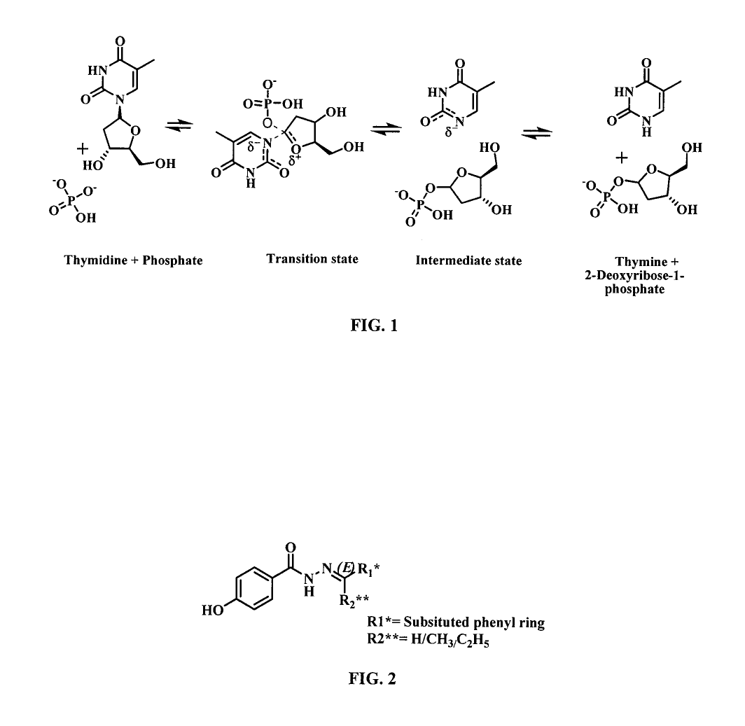

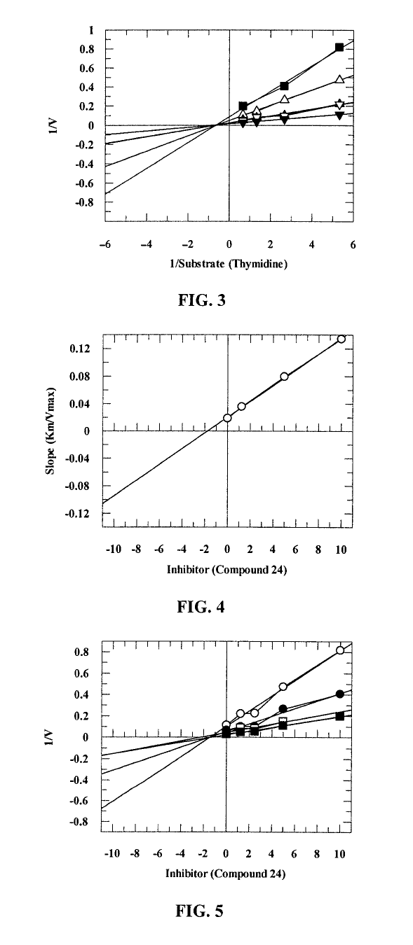

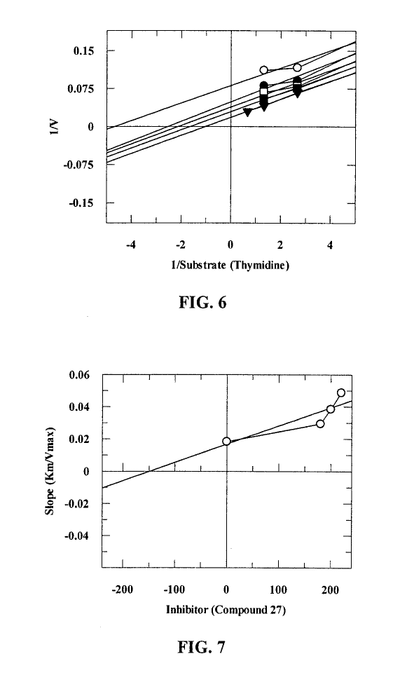

The reaction catalyzed by thymidine phosphorylase involves reversible phosphorolysis of thymidine (FIG. 1), to thymine and 2-deoxyribose-1-phosphate (Bronckaers et al., 2009).

### Protocol

Reaction mixture contained 150 μL of potassium phosphate buffer (pH 7.0, 50 mM), 20 L of enzyme (0.058 U/well) and 10 μL of test compound (0.5 mM in dimethylsulfoxide). The reaction mixture was incubated for 10 min at 30° C. 20 μL of substrate (1.5 mM) was then added and change in absorbance was observed for 10 minutes at 290 nm in ELISA plate reader (Spectramax, Molecular Devices, CA, USA). 7-Deazaxanthine was used as a positive control.

### Calculations of Inhibitory Activities

The enzyme inhibitory activities were calculated using the following formula:

Percent Inhibition=100−(O.D. of test/O.D. of control)×100

Where test is the enzyme activity with sample, and control is the enzyme activity without sample, and O.D. is optical density.

### IC₅₀ Value Determination

The IC₅₀ of the compounds was evaluated by monitoring the inhibitory effect of different concentrations ranging from 2-500 μM on the conversion of thymdine to thymine. The IC₅₀ of the compounds was calculated using EZ-Fit Enzyme Kinetic Program (Perrella Scientific Inc., Amhrest, U.S.A.).

### Mechanistic Studies

Kinetic studies were carried out to find the mechanism of inhibitor action. Inhibitor could bind with enzyme in multiple ways such as in competitive, non-competitive, mixed or uncompetitive way. In kinetic assay, the enzyme (0.058 U/200 μL) was incubated with different concentrations of inhibitor for 10 min at 30° C. The reactions was then initiated by adding different concentrations (0.1875 mM-1.5 mM) of substrate (thymidine) and the resulting degradation of thymidine was measured continuously at 290 nm for 10 min on a ELISA plate reader. Every experiment was run in triplicate.

### Determination of Type of Inhibition

Line-weaver Burk plot was plotted to determine the type of inhibition. This was accomplished by plotting the reciprocal of the rate of reaction against the reciprocal of the substrate concentration. Ki values were determined by secondary re-plot, and reconfirmed by Dixon plot. The Ki was determined by non-linear regression equation.

### MTT Assay

Anti-proliferative activity of active compounds was evaluated by using the standard MTT (3-[4,5-dimethylthiazole-2-yl]-2,5-diphenyl-tetrazolium bromide) colorimetric assay in 96-well plate (Dimas et al., 1998).

### Principle

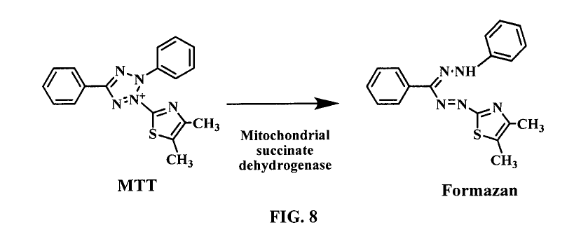

It is a colorimetric assay that measures the reduction of MTT, by mitochondrial enzyme i.e. succinate dehydrogenase. The MTT enters into the mitochondria of cell, where it is reduced to an insoluble formazan salt (FIG. 8). The extent of MTT reduction was measured at 540 nm. As reduction of MTT can only occur in metabolically active cells, the level of activity is actually a measure of the viability of the cells (Vega-Avila and Pugsley, 2011).

### Protocol

Mouse fibroblast cell line 3T3 was cultured in DMEM, supplemented with 5% of FBS, 100 IU/mL of penicillin and 100 μg/mL of streptomycin, and kept at 37° C. in 5% CO₂ incubator. For the preparation of cell culture, 100 μL/well of cell solution (5×10⁴ cells/mL) was added into 96-well plate. The plate was incubated for overnight, and fresh medium was added after the removal of old medium. The test compounds were also added in different concentrations into the plate and plate was again incubated for 48 h. After the completion of this incubation period, 200 μL MTT (0.5 mg/mL) was added and plate was again incubated for 4 h, after this final incubation 100 μL of DMSO was added to each well. The level of MTT reduction to formazan was evaluated by change in absorbance at 540 nm using a micro plate reader (Spectra Max plus, Molecular Devices, CA, USA). The anti-proliferative activity was recorded as concentration of the inhibitor causing 50% growth inhibition (IC₅₀) for 3T3, and PC3 cells.

1. A method for treating cancer in humans in need of treatment, administering a suitable dose of an inhibitor of thymidine phosphorylase enzymes selected from a group 4-hydroxybenzohydrazide derivatives consisting of:
4-Hydroxy-N′-[(E)-(2-hydroxyphenyl)methylidene]benzohydrazide;
4-Hydroxy-N′-[(E)-(3-hydroxyphenyl)methylidene]benzohydrazide;
4-Hydroxy-N′-[(E)-(4-hydroxyphenyl)methylidene]benzohydrazide;
4-Hydroxy-N′-[(E)-(2,3-dihydroxyphenyl)methylidene]benzohydrazide;
4-Hydroxy-N′-[(E)-(3,4-dihydroxyphenyl)methylidene]benzohydrazide;
4-Hydroxy-N′-[(E)-(2,4,5-trihydroxyphenyl)methylidene]benzohydrazide;
4-Hydroxy-N′-[(E)-(2-methoxyphenyl)methylidene]benzohydrazide;
4-Hydroxy-N′-[(E)-(4-methoxyphenyl)methylidene]benzohydrazide;
4-Hydroxy-N′-[(E)-(3,4-dimethoxyphenyl)methylidene]benzohydrazide;
4-Hydroxy-N′-[(E)-(4-chlorophenyl)methylidene]benzohydrazide;
4-Hydroxy-N′-[(E)-(3-chlorophenyl)methylidene]benzohydrazide;
4-Hydroxy-N′-[(E)-(4-bromophenyl)methylidene]benzohydrazide;
4-Hydroxy-N′-[(E)-(2,4-dichlorophenyl)methylidene]benzohydrazide;
4-Hydroxy-N′-[(E)-(2-methylphenyl)methylidene]benzohydrazide;
4-Hydroxy-N′-[(E)-(2-hydroxy-3-methoxyphenyl)methylidene]benzohydrazide;
4-Hydroxy-N′-[(E)-(2-hydroxy-5-methoxyphenyl)methylidene]benzohydrazide;
4-Hydroxy-N′-[(E)-(3-ethoxy-2-hydroxyphenyl)methylidene]benzohydrazide;
4-Hydroxy-N′-[(E)-(3,5-dibromo-2-hydroxyphenyl)methylidene]benzohydrazide;
4-Hydroxy-N′-[(E)-1-(2,6-dihydroxyphenyl)ethylidene]benzohydrazide;
4-Hydroxy-N′-[(E)-1-(2,5-dihydroxyphenyl)ethylidene]benzohydrazide;
4-Hydroxy-N′-[(E)-1-(2-hydroxy-3-methoxyphenyl)ethylidene]benzohydrazide; and
4-Hydroxy-N′-[(E)-1-(2,5-dihydroxyphenyl)propylidene]benzohydrazide
combined with suitable inert pharmaceutical ingredients.

2-3. (canceled)
